(19) United States
(12) Patent Application Publication
(10) Pub. No.: US 2011/0143378 A1
(43) Pub. Date: Jun. 16, 2011

(54) MICROFLUIDIC METHOD AND APPARATUS FOR HIGH PERFORMANCE BIOLOGICAL ASSAYS

(75) Inventor: Martin Andrew PUTNAM, Cheshire, CT (US)

(73) Assignee: CyVek LLC., Wallingford, CT (US)

(21) Appl. No.: 12/945,459

(22) Filed: Nov. 12, 2010

Related U.S. Application Data
(60) Provisional application No. 61/260,592, filed on Nov. 12, 2009.

(51) Int. Cl. G01N 33/543; C12M 1/34; B01L 3/00; G01N 21/00

(52) U.S. Cl. 435/7.92; 436/518; 435/287.2; 422/502; 422/82.05; 422/62

(57) ABSTRACT

The present invention provides a disposable microfluidic assay cartridge (1) which will contain at least one sample inlet well (2) that will feed into a microfluidic sub-unit (3) embedded within the disposable microfluidic assay cartridge (1). The microfluidic sub-unit (3) contains a series of microfluidic channels and micro-valves (4) that direct the sample from the sample inlet well (2) to separate and fluidicly-isolated reaction vessels (5) that contain encoded or non-encoded beads microparticles (6) which have been functionalized with a capture moiety or capture molecules such as antibodies, antigens, or oligomers. Assay reagents (7) including reagents R1, R2, R3, R4, such as labeled antibodies, will be introduced into the separate and fluidicly-isolated reaction vessels (5) via the series of microfluidic channels (8) and micro-valves (4). The series of microfluidic channels (8) and micro-valves (9) are provided to introduce reagents such as an enzymatic substrate (10) for producing a visible signal and a wash solution (11) to remove any non-specifically bound proteins or antibodies. The wash solution (11), along with non-specifically bound proteins or antibodies, is captured in an on-board waste receptacle (12). Chemical reactions taking place in the reaction vessels (5) are interrogated by a detection system (13).

### CROSS-REFERENCE TO RELATED APPLICATIONS

This application claims benefit to provisional patent application Ser. No. 61/260,592, filed 12 Nov. 2009, which is hereby incorporated by reference in its entirety.

### BACKGROUND OF THE INVENTION

1. Field of the Invention

The present invention relates to a method and apparatus for performing biological assays; and more particularly relates to a method and apparatus for performing biological assays using microfluidic technology.

2. Brief Description of Related Art

The primary factor affecting the data quality of a multiplexed system is biological cross reactivity, which is caused by mixing multiple analytes and a detection cocktail in a single reaction vessel. The mixing of analytes and the detection cocktail can result in unintended secondary reactions or interference that distort the measurements and severely compromise data quality. This biological cross reactivity can be mitigated by attempting to design the assay with components that do not negatively react; however, this becomes increasingly impractical and difficult (due to the high number of variables introduced) as the multiplex level increases. Moreover, for sets of antibodies in the assay with components that do not negatively react, the multiplexed result is still typically lower because the different environments being used will typically compromise the multiplexed result.

### SUMMARY OF THE INVENTION

The present invention provides a new and unique method and apparatus for performing a biological assay on a sample, which is summarized below with reference numerals consistent with that shown in FIGS. 1 and 2.

According to some embodiments of the present invention, the apparatus, such as (50) shown in FIG. 1, may take the form of a biological assay device or apparatus comprising: a microfluidic assay cartridge (1) that contains at least one sample inlet well (2) configured to receive a sample; and a microfluidic sub-unit (3) associated with the microfluidic assay cartridge (1) and comprising microfluidic channels (8), micro-valves (4, 4a, 9) and separate and fluidicly-isolated reaction vessels (5).

The separate and fluidicly-isolated reaction vessels (5) may be configured to contain the encoded or non-encoded beads or microparticles (6) which have been functionalized with a capture moiety or capture molecules.

The microfluidic channels (8) and micro-valves (4, 4a, 9) may be configured to respond to signaling containing information about performing the biological assay and to controllably receive the sample and a plurality of reagents in the separate and fluidicly-isolated reaction vessel (5), and to provide from the separate and fluidicly-isolated reaction vessels (5) light containing information about the biological assay performed on the sample by the encoded or non-encoded microparticles (6) as a result of the reagents.

In particular, the microfluidic channels (8) and micro-valves (4, 4a, 9) may be configured to respond to the signaling containing information about performing the biological assay and to introduce into the separate and fluidicly-isolated reaction vessels (5) the following:
assay reagents (7), including a plurality of reagents (R1, R2, R3, R4), including labeled antibodies,
reagents, including an enzymatic substrate (10), for producing a visible signal, and
a wash solution (11) to remove any non-specifically bound proteins or antibodies.

The separate and fluidicly-isolated reaction vessels (5) may be configured to allow chemical reactions to take place for performing the biological assay, and to provide visible light containing information about the biological assay performed to be interrogated, e.g., by a detection system (13).

According to some embodiments, the present invention may comprise one or more of the following features: The microfluidic sub-unit (3) may be configured to contain on-board the assay reagents (7), including the plurality of reagents (R1, R2, R3, R4), such as labeled antibodies. The microfluidic sub-unit (3) may be configured to contain on-board the reagents such as an enzymatic substrate (10) for producing the visible signal. The microfluidic sub-unit (3) may be configured to contain on-board the wash solution (11) to remove any non-specifically bound proteins or antibodies. Embodiments are also envisioned in which the assay reagents (7), the enzymatic substrate (10) or wash solution (11) are not contained on-board, but instead form part of another device, apparatus or equipment. The apparatus may comprise an on-board waste receptacle (12) that is configured to capture the wash solution (11), along with non-specifically bound proteins or antibodies. The microfluidic assay cartridge (1) may be disposable. The apparatus may comprise the detection system (13) configured to respond to the visible signal, and provide a signal containing information about the biological assay performed. The apparatus may comprise a controller (14) configured to execute a computer program code and to provide the signaling to each microfluidic channel (8) and micro-valves (4, 4a, 9) in order to perform the biological assay. Each of the series of microfluidic channels (8) may be configured to correspond to a respective one of the at least one sample inlet well (2). Embodiments for some biological assays are also envisioned in which the wash (10) is optional, and only the assay reagents (7) and the enzymatic substrate (10) are introduced, but not the wash (10). The separate and fluidicly-isolated reaction vessels (5) include channels C1, C2, C3, C4 that may be configured to conduct independent biological assays, where the channels C1, C2, C3, C4 are understood to be separate and fluidicly-isolated from one another so as to substantially eliminate biological cross reactivity between the biological assays performed in the respective channels C1, C2, C3, C4. The encoded or non-encoded beads or microparticles (6) contained in each channels C1, C2, C3, C4 may be functionalized with the same capture moiety or capture molecules; or the encoded or non-encoded beads or microparticles (6) contained in each channels C1, C2, C3, C4 may be each functionalized with a different capture moiety or capture molecules; or some combination thereof, where some of the channels C1, C2, C3, C4 may be functionalized with the same capture moiety or capture molecules, while others of the channels C1, C2, C3, C4 may be functionalized with a different same capture moiety or capture molecules.

According to some embodiments of the present invention, the apparatus, such as (50′) shown in FIG. 2, may also take the form of a disposable microfluidic assay cartridge (1) that contains at least one sample inlet well (2) and is configured so the at least one sample inlet well (2) will feed, e.g. based at least partly on some control logic, into a microfluidic sub-unit (3′) embedded within the disposable microfluidic assay cartridge (1); the microfluidic sub-unit (3′) may comprise microfluidic vessels or channels (5) and separate and fluidicly-isolated reaction vessels (16), where the disposable microfluidic sub-unit (3′) may be configured to direct the sample from the at least one sample inlet well (2) to an ensemble of differentiated microparticles (6′) that have been functionalized with a capture moiety or capture molecules such as antibodies, antigens, or oligomers, to segregate the ensemble of differentiated microparticles (6′) by type into separate microfluidic vessels or channels (5), where the separate and fluidicly-isolated reaction vessels (16) may be configured to isolate by type the ensemble of differentiated microparticles (6′), to allow chemical reactions to take place for performing the biological assay, and to provide visible light containing information about the biological assay performed to be interrogated, e.g., by a detection system (13′).

According to some embodiments of the present invention, the apparatus may take the form of a controller (14) that may be configured to control the performance of a biological assay by a biological assay device comprising a microfluidic assay cartridge (1) that contains at least one sample inlet well (2) configured to receive a sample; and a microfluidic sub-unit (3) associated with the microfluidic assay cartridge (1) and comprising microfluidic channels (8), micro-valves (4, 4a, 9) and separate and fluidicly-isolated reaction vessels (5), and the separate and fluidicly-isolated reaction vessels (5) containing encoded or non-encoded microparticles (6) which have been functionalized with a capture moiety.

In this embodiment, the controller (14) may comprise:

at least one processor and at least one memory including computer program code; the at one memory and the computer program code may be configured, with the at least one processor, to cause the controller (14) at least to provide signalling containing information about performing the biological assay to the microfluidic channels (8) and micro-valves (4, 9),
where the microfluidic channels (8) and micro-valves (4, 4a, 9) are configured to respond to the signaling, to direct the sample from the at least one sample inlet well (2) to the separate and fluidicly-isolated reaction vessels (5), and to introduce into the separate and fluidicly-isolated reaction vessels (5) a plurality of reagents, so as to provide from the separate and fluidicly-isolated reaction vessels (5) light containing information about the biological assay performed on the sample by the encoded or non-encoded microparticles (6) from the separate and fluidicly-isolated reaction vessels (5) as a result of the reagents.

The microfluidic channels (8) and micro-valves (4, 9) may be configured to respond to the signaling containing information about performing the biological assay and to introduce into the separate and fluidicly-isolated reaction vessels (5) the following:
assay reagents (7), including a plurality of reagents (R1, R2, R3, R4), such as labeled antibodies,
reagents, including an enzymatic substrate (10), for producing a visible signal, and
introduce a wash solution (11) to remove any non-specifically bound proteins or antibodies;

where the separate and fluidicly-isolated reaction vessels (5) may be configured to allow chemical reactions to take place for performing the biological assay, and to provide the visible light containing information about the biological assay performed to be interrogated, based at least partly on the signalling received.

According to some embodiments, the present invention may also take the form of a method for performing the biological assay process using a new and unique separation technique consistent with that set forth above. The method may be implemented by providing the means set forth above for automatically separating components where negative cross reactions may occur, and by employing the disposable microfluidic assay cartridge that will automate some of the manual steps typically associated with these types of tests. The separation technique set forth herein performing the biological assay process will substantially minimize the need to design around cross reactivity.

According to some embodiments, the present invention may also take the form of an apparatus for performing a biological assay on a sample comprising: a microfluidic assay cartridge (1) that contains at least one sample inlet well (2) configured to receive a sample; and a microfluidic sub-unit (3) associated with the microfluidic assay cartridge (1) and comprising microfluidic channels (8) and separate and fluidicly-isolated reaction vessels (5), the separate and fluidicly-isolated reaction vessels (5) configured to contain encoded or non-encoded microparticles (6) which have been functionalized with a capture moiety; where the microfluidic channels (8) is configured to respond to a control impulse containing information about performing the biological assay and to receive the sample and a plurality of reagents in the separate and fluidicly-isolated reaction vessels (5), and to provide from the separate and fluidicly-isolated reaction vessels (5) light containing information about the biological assay performed on the sample by the encoded or non-encoded microparticles (6) as a result of the reagents. By way of example, the control impulse may take the form of at least one control signal that opens or closes a micro-valve arranged in relation to the microchannel (8) that causes the sample and the plurality of reagents to flow into the separate and fluidicly-isolated reaction vessels (5) in order to perform the biological assay, or that causes a device arranged in relation to the microchannel (8) to provide positive or negative pressure in the microchannel (8) that causes the sample and the plurality of reagents to flow into the separate and fluidicly-isolated reaction vessels (5) in order to perform the biological assay.

Embodiments are also envisioned within the spirit of the present invention in which, instead of using functionalized encoded or non-encoded microparticles (6), the inside surface of the reaction vessel (5) may be functionalized, e.g. by coating, with the capture moiety or molecules, consistent with that disclosed in Ser. No. 61/263,572, filed 23 Nov. 2010, and hereby incorporated by reference in its entirety.

### ADVANTAGES

Some advantages of the embodiments of the present invention include substantially minimizing the need to design around cross reactivity by providing a means for automatically separating components where negative cross reactions occur. Additionally, this biological assay device will improve ease of use by employing a disposable microfluidic assay cartridge that will automate some of the manual steps typically associated with these types of tests. This biological assay device will optimize buffer conditions to produce independently optimized biological assays. The optimize buffer conditions may include optimizing in relation to the pH, salinity or both. This biological assay device will also allow samples to be independently diluted with buffer solution with respect to each channel.

It is the purpose of the present invention to deliver an apparatus or a method that provides multi-sample, multiplex biological assays with data quality that is significantly improved over current methods while at the same time providing greater ease of use.

### BRIEF DESCRIPTION OF THE DRAWING

The drawing, which are not necessarily drawn to scale, includes the following Figures:

FIG. 1, includes FIGS. 1(a) which shows a microfluidic assay cartridge according to some embodiments of the present invention, includes FIG. 1(b) which shows a microfluidic sub-unit corresponding to at least one sample inlet well of the microfluidic cartridge shown in FIG. 1(a) according to some embodiments of the present invention; and includes FIG. 1(c) which shows a flowchart having steps for performing a biological assay, e.g. using the combination of the microfluidic assay cartridge shown in FIG. 1(a) and the microfluidic sub-unit shown in FIG. 1(c).

FIG. 2, includes FIGS. 2(a) which shows a microfluidic assay cartridge (1) according to some embodiments of the present invention, and includes FIG. 2(b) which shows a microfluidic sub-unit (3′) according to some embodiments of the present invention.

### DETAILED DESCRIPTION OF THE INVENTION

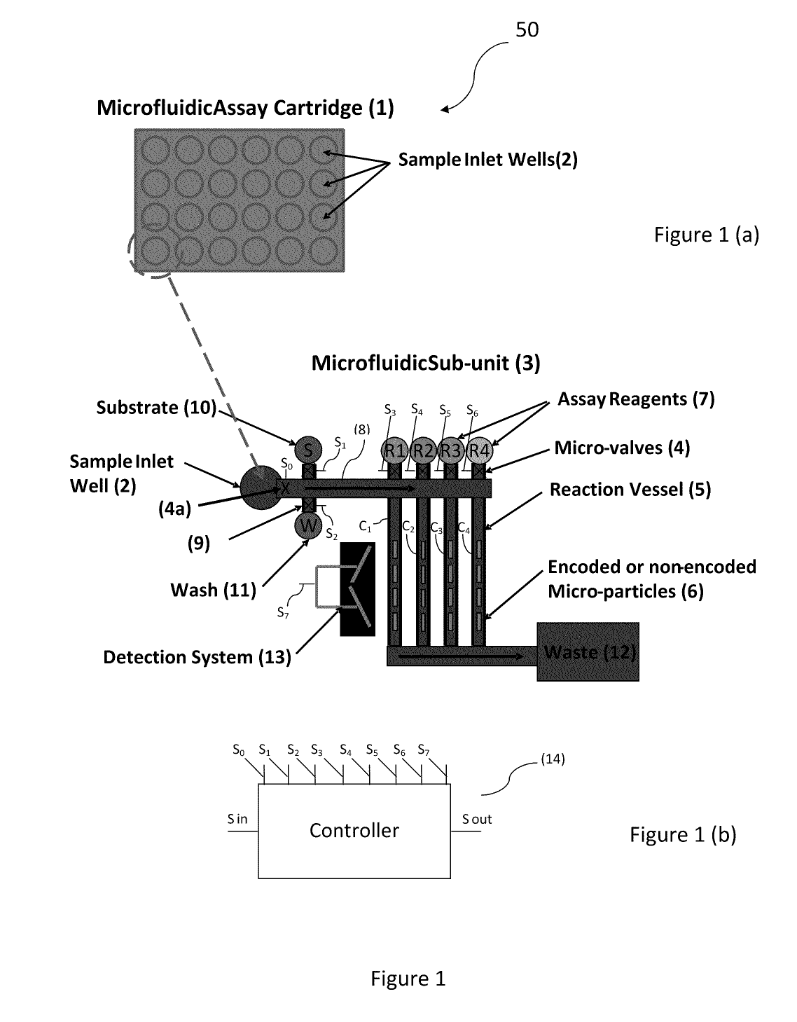

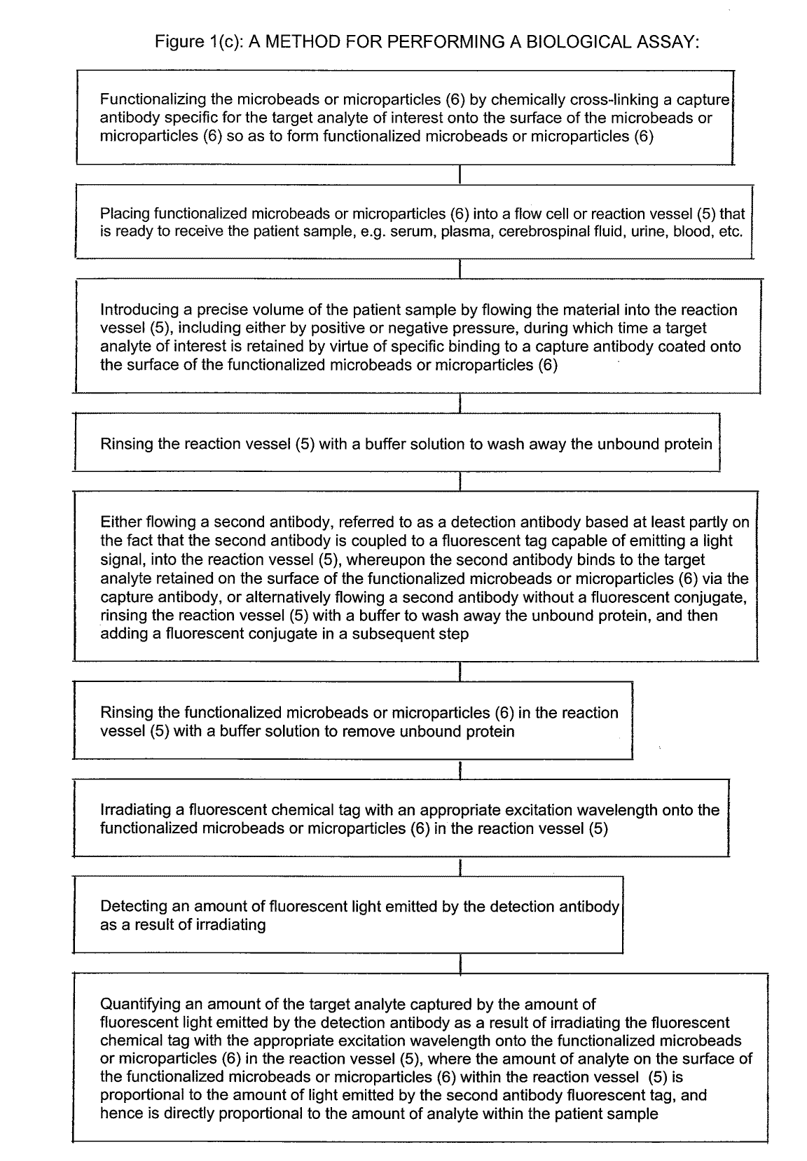

### FIG. 1

In FIG. 1, the present invention takes the form of an apparatus 50 shown in FIG. 1 that may include a microfluidic assay cartridge (1) which will contain at least one sample inlet well (2), as shown in FIG. 1(a). Each sample inlet well (2) will feed, e.g. based at least partly on some control logic, into a respective microfluidic sub-unit (3) embedded within the microfluidic assay cartridge (1), as shown in FIGS. 1 and 1(b). In FIG. 1(a), the microfluidic assay cartridge (1) is shown by way of example as having a plurality of sample inlet wells (2) in the form of 4 by 6 matrix, totally 24 sample inlet wells. The scope of the invention is not intended to be limited to the number of sample inlet wells (2), and is intended to include any number of sample inlet wells (2) ranging from 1 sample inlet well (2) to N sample inlet wells (2). The microfluidic assay cartridge (1) and/or microfluidic sub-unit (3) may be constructed and/or made from a material so as to be disposable or reusable, and the scope of the invention is not intended to be limited to the type or kind of material used to construct or make the microfluidic assay cartridge (1) and/or microfluidic sub-unit (3) either now known or later developed in the future.

The microfluidic sub-unit (3) contains a series of microfluidic channels and micro-valves (4) that direct the sample from the sample inlet well (2) to separate and fluidicly-isolated reaction vessels (5) that contain encoded or non-encoded microparticles (6) which have been functionalized with a capture moiety or capture molecules such as antibodies, antigens, or oligomers, as shown in FIG. 1(b). Assay reagents (7) including reagents R1, R2, R3, R4, such as labeled antibodies, will be introduced into the separate and fluidicly-isolated reaction vessels (5) via the series of microfluidic channels (8) and micro-valves (4). Additionally, the series of microfluidic channels (8) and micro-valves (9) are provided to introduce reagents such as an enzymatic substrate (10) for producing a visible signal and a wash solution (11) to remove any non-specifically bound proteins or antibodies. The wash solution (11), along with non-specifically bound proteins or antibodies, is captured in an on-board waste receptacle (12). Chemical reactions taking place in the reaction vessels (5) are interrogated by a detection system (13).

By way of example, the separate and fluidicly-isolated reaction vessels (5) may be configured to contain encoded or non-encoded microparticles (6) by necking down one end of the separate and fluidicly-isolated reaction vessels (5) so the encoded or non-encoded microparticles (6) cannot pass out of the separate and fluidicly-isolated reaction vessels (5). The scope of the invention is intended to includes other ways of configuring the separate and fluidicly-isolated reaction vessels (5) so as to contain encoded or non-encoded microparticles (6).

In FIG. 1, each of the at least one sample inlet well (2) of the disposable microfluidic assay cartridge (1) corresponds to a respective microfluidic sub-unit (3) embedded within the disposable microfluidic assay cartridge (1). However, the scope of the invention is also intended to include embodiments in which multiple sample inlet wells (2) of the disposable microfluidic assay cartridge (1) are configured to correspond to a respective microfluidic sub-unit (3) via, e.g., a manifold device. By way of example, a first column or group of four sample inlet wells (2) of the disposable microfluidic assay cartridge (1) may correspond to a first microfluidic sub-unit (3); a second column or group of four sample inlet wells (2) of the disposable microfluidic assay cartridge (1) may correspond to a second microfluidic sub-unit (3); . . . ; and a sixth column or group of four sample inlet wells (2) of the disposable microfluidic assay cartridge (1) may correspond to a sixth microfluidic sub-unit (3). Alternatively, by way of example, a first row or group of six sample inlet wells (2) of the disposable microfluidic assay cartridge (1) may correspond to a first microfluidic sub-unit (3); a second row or group of six sample inlet wells (2) of the disposable microfluidic assay cartridge (1) may correspond to a second microfluidic sub-unit (3); . . . ; and a fourth row or group of four sample inlet wells (2) of the disposable microfluidic assay cartridge (1) may correspond to a fourth microfluidic sub-unit (3). The scope of the invention is also intended to include embodiments in which all N sample inlet wells (2) of the disposable microfluidic assay cartridge (1), where N equals 24 as shown in FIG. 1 a, are configured to correspond to a single microfluidic sub-unit (3) via, e.g., a manifold device.

In FIG. 1, each assay reagent R1, R2, R3, R4 corresponds to, feeds into and is assigned a respective channel C1, C2, C3, C4 of the reaction vessels (5). However, the scope of the invention is also intended to include embodiments in which each assay reagent R1, R2, R3, R4 feeds into multiple channels C1, C2, C3, C4 of the reaction vessels (5).

In FIG. 1, each of the microfluidic sub-unit (3) embedded within the disposable microfluidic assay cartridge (1) has a respective detection system (13). However, the scope of the invention is also intended to include embodiments in which multiple microfluidic sub-unit (3) are configured to correspond to a respective detection system (13). By way of example, a first column or group of four microfluidic sub-unit (3) may correspond to a first detection system (13); a second column or group of four microfluidic sub-unit (3) may correspond to a second detection system (13); . . . ; and a sixth column or group of four microfluidic sub-unit (3) may correspond to a sixth detection system (13). Alternatively, by way of example, a first row or group of six microfluidic sub-unit (3) may correspond to a first detection system (13); a second row or group of six microfluidic sub-unit (3) may correspond to a second detection system (13); . . . ; and a fourth row or group of six microfluidic sub-unit (3) may correspond to a fourth detection system (13). The scope of the invention is also intended to include embodiments in which N microfluidic sub-unit (3), where N, e.g., equals 24 corresponding to that shown in FIG. 1, are configured to correspond to a single detection system (13). The scope of the invention is also intended to include embodiments in which the detection system (13) is on-board and forms part of microfluidic sub-unit (3), as well as embodiments where the detection system (13) is not on-board but forms part of another device, apparatus or equipment either now known or later developed in the future.

The apparatus may also include a controller (14) for implementing the functionality associated with the biological assay performed by the microfluidic sub-unit (3) embedded within the disposable microfluidic assay cartridge (1). The controller (14) may be configured to execute a computer program code and to provide the signaling along signal paths, e.g., S₀, S₁, S₂, S₃, S₄, S₅, S₆, to each microfluidic channel (8) and/or micro-valves (4, 9) in order to perform the biological assay. In operation, the controller (14) may be configured to execute the computer program code and to exchange signaling along signal path S₇ with the detection system (13), including receiving a detection system signal containing information about the chemical reactions taking place in the reaction vessels (5) being interrogated by the detection system (13). The controller (14) may also be configured to receive an input signal(s) along signal path Sᵢₙ, and to provide an output signal(s) along signal path Sₒᵤₜ. By way of example, the output signal along signal path Sₒᵤₜ may contain either the raw detection system signal containing information about the chemical reactions taking place in the reaction vessels (5) being interrogated by the detection system (13), or a processed detection system signal containing information about the chemical reactions taking place in the reaction vessels (5) being interrogated by the detection system (13). By way of example, the input signal along signal path Sᵢₙ may contain information to control or modify the functionality of the controller (14), including a signal requesting the provisioning of the output signal along signal path Sₒᵤₜ. The scope of the invention is not intended to be limited to the type or kind of information being provided to or received by the controller (14) via the input signal along signal path Sᵢₙ or the type or kind of information being provided from the controller (14) via the output signal along signal path Sₒᵤₜ either now known or later developed in the future. Further, by way of example, the controller (14) may be implemented using hardware, software, firmware, or a combination thereof. In a typical software implementation, the controller (14) would include one or more microprocessor-based architectures having a processor or microprocessor, memory such as a random access memory (RAM) and/or a read only memory (ROM), input/output devices and control, data and address buses connecting the same. A person skilled in the art would be able to program such a microcontroller or microprocessor-based implementation with the computer program code to perform the functionality described herein without undue experimentation. The scope of the invention is not intended to be limited to any particular microprocessor-based architecture implementation using technology either now known or later developed in the future.

Embodiments are envisioned in which the controller (14) either is on-board and forms part of the apparatus (50), or is not on-board but forms part of another apparatus, device, system or equipment that cooperates with the apparatus (50) in relation to implementing the biological assay process with the microfluidic technology disclosed herein.

In FIG. 1(a), the microfluidic sub-unit (3) is shown, by way of example, with micro-valves (4, 9) arranged in relation to the substrate (10), the wash (11) and the assay reagents (7) to control the introduction of the assay reagents to the reaction vessel (5) in response to the signalling along signalling paths S₁, S₂, S₃, S₄, S₅, S₆, using steps 2-8 described below and set forth in the flowchart shown in FIG. 1(c). Embodiments are also envisioned in which the micro-valves (4) provide information back to the controller (14) via corresponding signalling along signalling paths S₁, S₂, S₃, S₄, S₅, S₆. for controlling the introduction of the assay reagents (7), the substrate (10) and the wash (11) Embodiments are also envisioned in which other micro-valves are arranged at other points in relation to each microfluidic channel (8), e.g. such as micro-valves (4a) in FIG. 1(b) arranged in relation to the interface between each microfluidic channel (8) and the at least one sample inlet well (2) for controlling the provisioning of the sample into the microfluidic channel (8) with signalling along signal path S₀. Embodiments are also envisioned in which other micro-valves are arranged in relation to the reaction vessel (5), including at either or both ends, so as to control the passage of the particles (6) through the reaction vessel (5). The scope of the invention is not intended to be limited to the number, position, or arrangements of the micro-valves, like (4) or (4a) or (9).

By way of example, the micro-valves (4, 4a, 9), microparticles (6), detection system (13), along with other components or devices shown and described herein in relation to FIG. 1, are either known in the art, or can be implemented to perform the desired functionality without undue experimentation by one skilled in the art; and the scope of the invention is not intended to be limited to any particular type or kind thereof either now known or later developed in the future. Furthermore, based of the disclosure herein, one skilled in the art could implement the apparatus 50 shown in FIG. 1, including the disposable microfluidic assay cartridge (1) shown in FIG. 1(a) and the microfluidic sub-unit (3) embedded therein shown in FIG. 1(b), to perform the desired functionality without undue experimentation.

The present invention is described by way of using micro-valves configured to control the flow of one or more of the sample, the assay reagents (7), the substrate (10) and the wash (13) into the separate and fluidicly-isolated reaction vessels (5). However, the scope of the invention is intended to include using other types or kind of techniques either now known or later developed in the future to control the flow of one or more of the sample, the assay reagents (7), the substrate (10) and the wash (13) into the separate and fluidicly-isolated reaction vessels (5), e.g., such as by using a configuration to provide positive pressure to push and cause the flow of one or more of the sample, the assay reagents (7), the substrate (10) and the wash (13) into the separate and fluidicly-isolated reaction vessels (5), or such as by using a configuration to provide negative pressure (e.g. a vacuum) to pull (or draw) and cause the flow of one or more of the sample, the assay reagents (7), the substrate (10) and the wash (13) into the separate and fluidicly-isolated reaction vessels (5), or such as by using some combination of pushing and/or pulling to cause the flow of one or more of the sample, the assay reagents (7), the substrate (10) and the wash (13) into the separate and fluidicly-isolated reaction vessels (5). The configuration to provide positive pressure may be configured on the upper end (as shown in FIG. 1(b)) of the separate and fluidicly-isolated reaction vessels (5) in relation to the assay reagents (7) and channels C1, C2, C3, C4, while the configuration to provide negative pressure may be configured on the lower end (as shown in FIG. 1(b)) of the separate and fluidicly-isolated reaction vessels (5) in relation to the waste (12) and channels C1, C2, C3, C4.

### Immunoassay Process for Sandwich ELISAs

The process of conducting an immunoassay in a cartridge according to the present invention using a sandwich enzyme-linked immunosorbent assay (ELISA) entails the following steps:

Step 1. A capture antibody specific for the target analyte of interest is chemically cross-linked onto the surface of the microbeads or microparticles (6) so as to form functionalized microbeads or microparticles (6).

Step 2. The functionalized microbeads or microparticles (6) once placed into the flow cell or reaction vessel (5) is then ready to receive, e.g., a patient sample (serum, plasma, cerebrospinal fluid, urine, blood, etc).

Step 3. A precise volume of the patient sample is then introduced by flowing the material into the reaction vessel (5) either, e.g., by positive or negative pressure, during which time the target analyte of interest is retained by virtue of specific binding to the capture antibody coated onto the surface of functionalized microbeads or microparticles (6).

Step 4. The reaction vessel (5) is then rinsed with a buffer to substantially wash away unbound protein.

Step 5. The second antibody, referred to as a detection antibody since it is coupled to a fluorescent tag capable of emitting a light signal, is then flowed into the reaction vessel (5) whereupon it binds to the target analyte retained on the surface of the functionalized microbeads or microparticles (6) via the capture antibody.

Step 5a. An alternative embodiment of this process is to use a second antibody without a fluorescent conjugate, and then to add the fluorescent conjugate in a subsequent step. Note that this may also include an additional rinse step prior to adding the fluorescent conjugate.

Step 6. After step 5, the functionalized microbeads or microparticles (6) in the reaction vessel (5) is then rinsed again with a buffer to remove unbound protein.

Step 7. The amount of the target analyte captured is quantified by the amount of fluorescent light emitted by the detection antibody as a result of irradiating the fluorescent chemical tag with the appropriate excitation wavelength onto the functionalized microbeads or microparticles (6) in the reaction vessel (5).

Step 8. The amount of analyte on the surface of the functionalized microbeads or microparticles (6) within the reaction vessel (5) is proportional to the amount of light emitted by the second antibody fluorescent tag, and hence is directly proportional to the amount of analyte within the patient sample.

The controller (14) shown in FIG. 1(b) may be implemented and configured to provide the signalling to perform the biological assay using steps 2-8 set forth above.

The scope of the invention is by way of example using the sandwich ELISA biological assay technique. However, the scope of the invention is not intended to be limited to using the sandwich ELISA biological assay technique, e.g., embodiments are also envisioned using other types or kind of biological assay techniques either now known or later developed in the future, including an “indirect” ELISA, a competitive ELISA, a reverse ELISA, as well as other non-ELISA techniques.

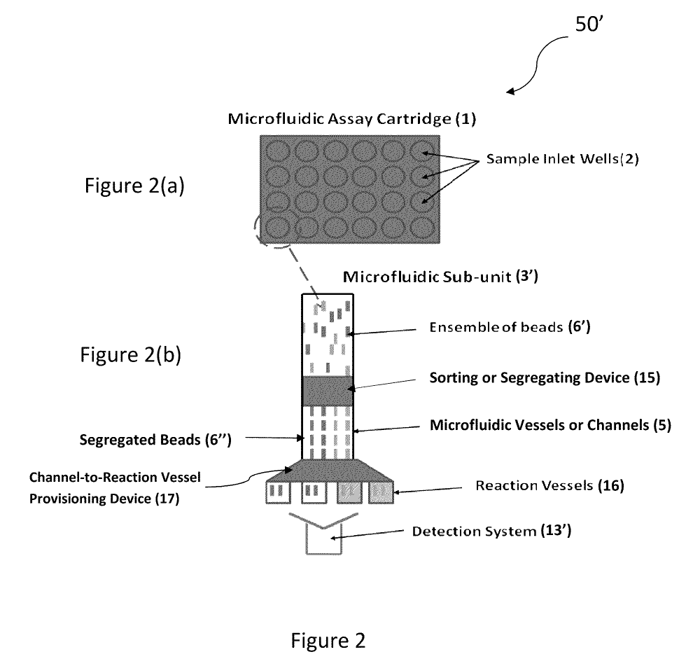

### FIG. 2

The present invention may also take the form of an apparatus 50′ shown in FIG. 2 that may include a microfluidic assay cartridge (1) as shown in FIG. 2(a), which will contain at least one sample inlet well (2). Each sample inlet well (2) will feed into a respective microfluidic sub-unit (3′) embedded within the microfluidic assay cartridge (1). The microfluidic sub-unit (3′) may be configured to direct the sample from the sample inlet well (2) in the form of an ensemble of differentiated microparticles (6′) that have been functionalized with a capture moiety or capture molecules such as antibodies, antigens, or oligomers. The differentiated microparticles (6′) are segregated, e.g. by a sorting or segregation device (15), by type into separate microfluidic channels (5) so as to take the form of segregated differentiated microparticles (6″), which are then isolated by type in separate and fluidicly-isolated reaction vessels (16), e.g., by a channel-to-reaction vessel provisioning device (17). Chemical reactions taking place in the reaction vessels (16) are interrogated by a detection system (13′).

Devices, such as the sorting or segregation device (15), channel-to-reaction vessel provisioning device (17) and detection system (13′) are either known in the art, or can be implemented to perform the desired functionality without undue experimentation by one skilled in the art; and the scope of the invention is not intended to be limited to any particular type or kind thereof either now known or later developed in the future.

Based of the disclosure herein, one skilled in the art could implement the apparatus 50′ shown in FIG. 2, including the microfluidic assay cartridge (1) shown in FIG. 2(a) and the microfluidic sub-unit (3′) embedded therein shown in FIG. 2(b), to perform the desired functionality without undue experimentation.

### Method for Performing a Biological Assay Using a Separation Technique

The present invention may also take the form of a method for performing the biological assay process using a new and unique separation technique consistent with that set forth above. The method may be implemented by providing the means set forth above for automatically separating components where negative cross reactions occur, and by employing the disposable microfluidic assay cartridge that will automate some of the manual steps typically associated with these types of tests. The separation technique set forth herein for performing the biological assay process will eliminate the need to design around cross reactivity.

By way of example, the method for performing a biological assay may be implemented using the microfluidic technology in FIG. 1 as follows:

providing a microfluidic assay cartridge (1) that contains at least one sample inlet well (2) configured to receive a sample; and a microfluidic sub-unit (3) associated with the microfluidic assay cartridge (1) and configured to controllably receive the sample from the microfluidic assay cartridge (1); the microfluidic sub-unit (3) comprising microfluidic channels (8), micro-valves (4, 4a, 9) and separate and fluidicly-isolated reaction vessels (5), the separate and fluidicly-isolated reaction vessels (5) containing encoded or non-encoded microparticles (6) which have been functionalized with a capture moiety or capture molecules;

responding to signaling containing information about performing the biological assay with the microfluidic channels (8) and micro-valves (4, 9), and controllably receiving the sample and the plurality of reagents in the separate and fluidicly-isolated reaction vessels (5), so as to provide light containing information about the biological assay performed on the sample by the encoded or non-encoded microparticles (6) as a result of the reagents. The method may also comprise responding to the signaling containing information about performing the biological assay with the microfluidic channels (8) and micro-valves (4, 9) and introducing into the separate and fluidicly-isolated reaction vessels (5) the following:
assay reagents (7), including a plurality of reagents (R1, R2, R3, R4), such as labeled antibodies,
reagents, including an enzymatic substrate (10), for producing a visible signal, and
a wash solution (11) to remove any non-specifically bound proteins or antibodies; and

allowing with the separate and fluidicly-isolated reaction vessels (5) chemical reactions to take place for performing the biological assay, and providing the visible light containing information about the biological assay performed to be interrogated, e.g. by the detection system (13).

Further, by way of example, the method for performing a biological assay may also be implemented using the microfluidic technology in FIG. 2.

Furthermore, by way of example, the method for performing a biological assay may also be implemented using the steps set forth above, including those set forth in relation to FIG. 1(c).

### The Microfluidic Technology

By way of example, the term “microfluidics” is generally understood to mean or deal with the behavior, precise control and manipulation of fluids that are geometrically constrained to a small, typically sub-millimeter, scale. In the present application, the microfluidic technology described herein is intended to include technology dimensioned in a range of about 20 micron to about 1000 microns, although the scope of the invention is not intended to be limited to any particular range.

### The Scope of the Invention

Embodiments shown and described in detail herein are provided by way of example only; and the scope of the invention is not intended to be limited to the particular configurations, dimensionalities, and/or design details of these parts or elements included herein. In other words, a person skilled in the art would appreciate that design changes to these embodiments may be made and such that the resulting embodiments would be different than the embodiments disclosed herein, but would still be within the overall spirit of the present invention.

It should be understood that, unless stated otherwise herein, any of the features, characteristics, alternatives or modifications described regarding a particular embodiment herein may also be applied, used, or incorporated with any other embodiment described herein. Also, the drawing herein are not drawn to scale.

Although the invention has been described and illustrated with respect to exemplary embodiments thereof, the foregoing and various other additions and omissions may be made therein and thereto without departing from the spirit and scope of the present invention.

What I claim is:

1. An apparatus for performing a biological assay on a sample comprising:
a microfluidic assay cartridge (1) that contains at least one sample inlet well (2) configured to receive a sample; and
a microfluidic sub-unit (3) associated with the microfluidic assay cartridge (1) and comprising microfluidic channels (8), micro-valves (4, 4a, 9) and separate and fluidicly-isolated reaction vessels (5);
the separate and fluidicly-isolated reaction vessels (5) configured to contain encoded or non-encoded microparticles (6) which have been functionalized with a capture moiety;
the microfluidic channels (8) and micro-valves (4, 4a, 9) configured to respond to signaling containing information about performing the biological assay and to controllably receive the sample and a plurality of reagents in the separate and fluidicly-isolated reaction vessels (5), and to provide from the separate and fluidicly-isolated reaction vessels (5) light containing information about the biological assay performed on the sample by the encoded or non-encoded microparticles (6) as a result of said reagents.

2. An apparatus according to claim 1, wherein the microfluidic channels (8) and micro-valves (4, 4a, 9) are configured to respond to the signaling containing information about performing the biological assay and to introduce into the separate and fluidicly-isolated reaction vessels (5) the following:
assay reagents (7), including a plurality of assay reagents (R1, R2, R3, R4), including labeled antibodies,
reagents, including an enzymatic substrate (10), for producing visible signal, and
a wash solution (11) to remove any non-specifically bound proteins or antibodies; and
the separate and fluidicly-isolated reaction vessels (5) configured to allow chemical reactions to take place for performing the biological assay.

3. An apparatus according to claim 1, wherein the microfluidic sub-unit (3) is configured to contain the assay reagents (7), including the plurality of reagents (R1, R2, R3, R4), such as labeled antibodies; and/or wherein the microfluidic sub-unit (3) is configured to contain the reagents such as an enzymatic substrate (10) for producing the visible signal.

4. An apparatus according to claim 1, wherein the microfluidic sub-unit (3) is configured to contain the wash solution (11) to remove any non-specifically bound proteins or antibodies.

5. An apparatus according to claim 1, wherein the apparatus comprises an on-board waste receptacle (12) that is configured to capture the wash solution (11), along with non-specifically bound proteins or antibodies.

6. An apparatus according to claim 1, wherein the microfluidic assay cartridge (1) is disposable.

7. An apparatus according to claim 1, wherein the apparatus comprises a detection system (13) configured to respond to the visible signal, and provide a detection system signal containing information about the biological assay performed.

8. An apparatus according to claim 1, wherein the apparatus comprises a controller configured to execute a computer program code and to provide the signaling to the microfluidic channels (8) and micro-valves (4, 4a, 9) in order to perform the biological assay.

9. An apparatus according to claim 1, wherein each of the plurality of microfluidic channels (8) corresponds to a respective one of the at least one sample inlet well (2).

10. An apparatus for performing a biological assay comprising:
a disposable microfluidic assay cartridge (1) that contains at least one sample inlet well (2) and is configured so said at least one sample inlet well (2) will feed, including based at least partly on some control logic, into a microfluidic sub-unit (3′) embedded within the disposable microfluidic assay cartridge (1);
the microfluidic sub-unit (3′) comprising microfluidic vessels or channels (5) and separate and fluidicly-isolated reaction vessels (16);
the disposable microfluidic sub-unit (3′) configured to direct a sample from said at least one sample inlet well (2) in the form of to an ensemble of differentiated microparticles (6′) that have been functionalized with capture molecules such as antibodies, antigens, or oligomers, to segregate the ensemble of differentiated microparticles (6′) by type into separate microfluidic channels (5) so as to form segregated or sorted differentiated microparticles (6″); and
the separate and fluidicly-isolated reaction vessels (16) configured to isolate by type the segregated or sorted differentiated microparticles (6″) to allow chemical reactions to take place for performing the biological assay, and to provide the visible light containing information about the biological assay performed to be interrogated, including to be interrogated by a detection system (13).

11. A controller configured to control the performance of a biological assay by a biological assay apparatus or device comprising a microfluidic assay cartridge (1) that contains at least one sample inlet well (2) configured to receive a sample; and a microfluidic sub-unit (3) associated with the microfluidic assay cartridge (1) and comprising microfluidic channels (8), micro-valves (4, 4a, 9) and separate and fluidicly-isolated reaction vessels (5), the separate and fluidicly-isolated reaction vessels (5) containing encoded or non-encoded microparticles (6) which have been functionalized with a capture moiety, the controller comprising:
at least one processor and at least one memory including computer program code;
the at one memory and the computer program code configured, with the at least one processor, to cause the controller at least to provide signalling containing information about performing the biological assay to the microfluidic channels (8) and micro-valves (4, 4a, 9),
where the microfluidic channels (8) and micro-valves (4, 4a, 9) are configured to respond to the signaling, to direct the sample from said at least one sample inlet well (2) to the separate and fluidicly-isolated reaction vessels (5), and to introduce into the separate and fluidicly-isolated reaction vessels (5) a plurality of reagents, so as to provide from the separate and fluidicly-isolated reaction vessels (5) light containing information about the biological assay performed on the sample by the encoded or non-encoded microparticles (6) from the separate and fluidicly-isolated reaction vessels (5) as a result of said reagents.

12. A controller according to claim 11, wherein the at one memory and the computer program code is configured, with the at least one processor, to cause the controller at least to receive a detection system signal containing information about visible light and to provide an output controller signal containing information about the biological assay performed and interrogated.

13. A controller according to claim 11, wherein the signalling provided from the controller causes the microfluidic channels (8) and micro-valves (4, 4a, 9) to introduce into the separate and fluidicly-isolated reaction vessels (5) the following:
assay reagents (7), including the plurality of assay reagents (R1, R2, R3, R4), including labeled antibodies,
reagents, including an enzymatic substrate (10), for producing visible signal, and
a wash solution (11) to remove any non-specifically bound proteins or antibodies; and
the separate and fluidicly-isolated reaction vessels (5) configured to allow chemical reactions to take place for performing the biological assay.

14. A method for performing a biological assay on a sample comprising:
providing a microfluidic assay cartridge (1) that contains at least one sample inlet well (2) configured to receive a sample; and a microfluidic sub-unit (3) associated with the microfluidic assay cartridge (1) and configured to controllably receive said sample from said microfluidic assay cartridge (1); the microfluidic sub-unit (3) comprising a plurality of microfluidic channels (8), micro-valves (4, 4a, 9) and separate and fluidicly-isolated reaction vessels (5), the separate and fluidicly-isolated reaction vessels (5) containing encoded or non-encoded microparticles (6) which have been functionalized with a capture moiety or capture molecules;
responding to signaling containing information about performing the biological assay with the microfluidic channels (8) and micro-valves (4, 4a, 9), and controllably receiving the sample and the plurality of reagents in the separate and fluidicly-isolated reaction vessels (5), so as to provide light containing information about the biological assay performed on the sample by the encoded or non-encoded microparticles (6) as a result of said reagents.

15. A method according to claim 14, wherein the method further comprises
responding to the signaling containing information about performing the biological assay with the microfluidic channels (8) and micro-valves (4, 4a, 9) and introducing into the separate and fluidicly-isolated reaction vessels (5) the following:
assay reagents (7), including a plurality of assay reagents (R1, R2, R3, R4), such as labeled antibodies,
reagents, including an enzymatic substrate (10), for producing a visible signal, and
a wash solution (11) to remove any non-specifically bound proteins or antibodies; and
allowing with the separate and fluidicly-isolated reaction vessels (5) chemical reactions to take place for performing the biological assay, and providing visible light containing information about the biological assay performed to be interrogated, including by a detection system (13).

16. A method according to claim 14, wherein the method further comprises detecting the visible light containing information about the biological assay performed and interrogated, and providing a detection system signal containing information about the visible light.

17. A method for performing a biological assay comprising:
placing functionalized microbeads or microparticles (6) into a flow cell or reaction vessel (5) that is ready to receive the patient sample, including serum, plasma, cerebrospinal fluid, urine, blood, etc;
introducing a precise volume of the patient sample by flowing the material into the reaction vessel (5), including either by positive or negative pressure, during which time a target analyte of interest is retained by virtue of specific binding to a capture antibody coated onto the surface of the functionalized microbeads or microparticles (6);
rinsing the reaction vessel (5) with a buffer solution to wash away the unbound protein;
either flowing a second antibody, referred to as a detection antibody based at least partly on the fact that the second antibody is coupled to a fluorescent tag capable of emitting a light signal, into the reaction vessel (5), whereupon the second antibody binds to the target analyte retained on the surface of the functionalized microbeads or microparticles (6) via the capture antibody, or alternatively flowing a second antibody without a fluorescent conjugate, rinsing the reaction vessel (5) with a buffer to wash away the unbound protein, and then adding a fluorescent conjugate in a subsequent step;
rinsing the functionalized microbeads or microparticles (6) in the reaction vessel (5) with a buffer solution to remove unbound protein;
irradiating a fluorescent chemical tag with an appropriate excitation wavelength onto the functionalized microbeads or microparticles (6) in the reaction vessel (5);
detecting an amount of fluorescent light emitted by the detection antibody as a result of irradiating;
quantifying an amount of the target analyte captured by the amount of fluorescent light emitted by the detection antibody as a result of irradiating the fluorescent chemical tag with the appropriate excitation wavelength onto the functionalized microbeads or microparticles (6) in the reaction vessel (5), where the amount of analyte on the surface of the functionalized microbeads or microparticles (6) within the reaction vessel (5) is proportional to the amount of light emitted by the second antibody fluorescent tag, and hence is directly proportional to the amount of analyte within the patient sample.

18. A method according to claim 17, wherein the method further comprises:
functionalizing the microbeads or microparticles (6) by chemically cross-linking a capture antibody specific for the target analyte of interest onto the surface of the microbeads or microparticles (6) so as to form functionalized microbeads or microparticles (6).

19. An apparatus for performing a biological assay on a sample comprising:
a microfluidic assay cartridge (1) that contains at least one sample inlet well (2) configured to receive a sample; and
a microfluidic sub-unit (3) associated with the microfluidic assay cartridge (1) and comprising microfluidic channels (8) and separate and fluidicly-isolated reaction vessels (5), the separate and fluidicly-isolated reaction vessels (5) configured to contain encoded or non-encoded microparticles (6) which have been functionalized with a capture moiety;
the microfluidic channels (8) configured to respond to a control impulse containing information about performing the biological assay and to receive the sample and a plurality of reagents in the separate and fluidicly-isolated reaction vessels (5), and to provide from the separate and fluidicly-isolated reaction vessels (5) light containing information about the biological assay performed on the sample by the encoded or non-encoded microparticles (6) as a result of said reagents.

20. An apparatus according to claim 19, wherein the control impulse takes the form of at least one control signal that opens or closes a microvalve arranged in relation to the microchannel (8) that causes the sample and the plurality of reagents to flow into the separate and fluidicly-isolated reaction vessels (5) in order to perform the biological assay, or that causes a device arranged in relation to the microchannel (8) to provide positive or negative pressure in the microchannel (8) that causes the sample and the plurality of reagents to flow into the separate and fluidicly-isolated reaction vessels (5) in order to perform the biological assay.
